(19) United States
(12) Patent Application Publication
(10) Pub. No.: US 2018/0268944 A1
(43) Pub. Date: Sep. 20, 2018

(54) SYSTEM, APPARATUS AND METHOD FOR MANAGEMENT OF HEALTH AND WELLNESS INFORMATION, AND MANAGEMENT OF TRANSACTIONS USING SAME

(71) Applicant: Ramkrishna Prakash, Austin, TX (US)

(72) Inventor: Ramkrishna Prakash, Austin, TX (US)

(21) Appl. No.: 15/926,635

(22) Filed: Mar. 20, 2018

Related U.S. Application Data
(60) Provisional application No. 62/473,951, filed on Mar. 20, 2017.

(51) Int. Cl. G16H 80/00; G16H 10/60; G16H 15/00

(52) U.S. Cl. CPC G16H 80/00; G16H 50/20; G16H 15/00; G16H 10/60

(57) ABSTRACT

Disclosed subject matter includes methods, systems and apparatus for management of health and wellness information.

### CROSS-REFERENCE TO RELATED APPLICATIONS

This application is related to provisional application No. 62/473,951 filed Mar. 20, 2017, (the “Parent Provisional”) the contents of which is expressly incorporated herein in its entirety.

This application claims priority to the Parent Provisional and hereby claims benefit of the filing dates thereof pursuant to 35 U.S.C. 119(e).

### FIELD OF THE INVENTION

This disclosure relates to systems, apparatus and methods for management of health and wellness information, and management of health and wellness information transactions.

### BACKGROUND

The recent trends in Accountable Care based payment models have necessitated the adoption of new process for care delivery that requires the co-ordination of a “network” of care providers who can engage in shared risk contracts. In addition, the need for sharing in the savings generated equitably is key to encourage the network providers to invest in improved care paradigms. Current approaches to digitize healthcare focus on improvement of operational efficiency, like electronic records and care collaboration software. However, these approaches are still based on the classical centralized authorization model that results in significant expense in implementation. These approaches are fundamentally limited in their ability to fully capitalize on the peer-to-peer digital workflow revolution that is sweeping other segments of industry like media, e-retail, etc.

The last decade has seen a significant change in health care ranging from a dramatic shift in the payment method from a “pay-for-service” model to “outcome based” model and to a focus on population “wellness” from a focus on “specialized” procedures. This new payment model based on effective care along with a focus on healthy living, called the “Accountable Care” paradigm, outlines the “new” goal for delivery of healthcare in the US. This realignment from a “procedure” based focus to “holistic care of the individual” necessitates that care providers form “networks” that work together towards a common goal of improving the care outcome of patients under care, for post-Acute Care episodes, or to provide sustained wellness between Acute Care episodes. The need for cooperation between care providers ranging from specialist to primary care physician, post-acute care providers to wellness providers (like nutritionist and rehabilitation nurses) has resulted in increasing digitization of patient care data in order to seamlessly communicate patient data between these multitudes of parties. Over the last decade this has led to increased adoption of Electronic Health Records (EHRs) systems as well as development of care collaboration software that enables the co-ordination of care across the various care providers. Though these solutions have significantly improved the tracking and efficiency for delivering care, they have resulted in creating islands of information. Hence, coordination of information between these systems has presented a significant challenge causing the delay of both the adoption of this new healthcare paradigm as well as posed serious challenges for health systems in developing scalable “networks” of providers.

One problem is that there has been a lot of health and wellness related data that has been collected by care providers and individuals but it has not been converted into consumable formats, with appropriate specific context, that enable a comprehensive individualized care plan that contributes to effective long term patient wellness. This stems from the key issue that most of this data is trapped in silos controlled by an individual care provider and is not readily accessible by their “network” of partners engaged in the care of the patient.

Furthermore, the accountability and authorization for accessing and modifying a given patient's data is limited to these individual silos. This results in each organization “modifying” its copy of patient data based on their independent interaction with the patient. This has led to the “network” of care providers having the task of constantly “updating” the patient profile, always trying to catch up to the illusive “latest valid profile” of the patient. This has been exacerbated by a tendency to allow for minimal authorization for the individual whose data is being modified to provide their own input, leading to erroneous information being introduced into their records resulting is both clinical and economic woes. This in turn leads to further challenges, as patients feel that they do not have an appropriate access and incentives to engage in their own care management, leading to a frustrating experience for both providers and patients. This has led to a breakdown in the overall accountability of all parties involved in yielding optimal care outcomes.

Another problem stemming from the construction of the current healthcare system is that providers in the healthcare industry are very weary of whether the data being used for clinical prescription is “accurate” as they expose themselves to significant liabilities unlike other industries if they are found to have made an error. Therefore, they are insistent on “appropriate validation” of the generation of data to ensure that they are not exposed to any liabilities stemming from erroneous information. Hence there is an inherent averseness towards using information that a provider has not collected itself or has been deemed reliable by a “liable” participant in their network. This has resulted in “forced aggregation” of health care data which in turn has led to increased costs and delays in care delivery, while still not preventing or uncovering data errors. The standard approach, adopted is by the dominant provider mandating that their “network” partners enter information they collect into its system which is then the “golden record” for the patient, which they allow other to only view. While, this avoids some liability issues it does not address the fact that the network providers need information in a timely manner. This problem is exacerbated for chronic patients with two or more issues which may be managed independently and contemporaneously by different providers and has led to a crisis in delivering coordinated care for these patients.

An additional issue in ensuring effective health care delivery is the accountability associated with who has accessed and reviewed the data, authorized changes, and finally executed care delivery. As most of the healthcare EHR systems were built to address a single domain of care providers they were designed for only one “key” individual to access and authorize changes therein. This was adequate when most health care providers delivered comprehensive care for an individual within a single provider system, which gathered all the data from their “client,” the patient. However, with the emerging trend wherein data is collected and processed by a multitude of providers and intermediaries like labs, technicians, home health care workers, patient, or even family members, this approach is limiting. Furthermore, with the formation of Accountable Care networks, wherein penalties for bad outcomes are high, resulting in non-collaborative behavior, effective automation of these care coordination capabilities is imperative. Finally, in the emerging Accountable Care landscape, compensation may be based on how effectively the network of providers work together to ensure improvement in the quality of care outcome while at the same time reducing associated costs. Hence, to truly incentivize different participants in the network to work together to pro-actively create better care regimes, there needs to be a merit based compensation of shared savings. To effectively allocate a proportionate share of such savings to the provider in the network that contributed the most towards the overall savings, a clear tracking of their contribution is vital. Without the ability for such tracking, a “least effort” approach by all providers in the network could occur, resulting in both an overall loss of income for care providers, and an adverse effect in quality of patient care.

As the Care Delivery Model is shifting to “outcome based” accountable care, there is an increasing need for the patient data to move “fluidly” across various approved care providers in the care network without sacrificing the privacy of the patient data. However, the single domain nature of EHR systems, which limits the portability of health data has resulted in significant challenges. Hence, providers often mandate that patients sign a HIPAA waiver to ensure timely care can be delivered to them. This has led to the leakage of patient health information resulting in unscrupulous providers targeting patients during need for medical care, one of their most vulnerable times.

This problem is made worse due to the fact that upon receiving this wavier information, it is frequently transferred via paper copies, leading to this vital information tending to linger a long time in the caregiver community. This has led to persistent fraud practices that adversely affect both payors and patients.

Though there have been many efforts via the Health Information Exchanges (HIE) to address the portability of this information across providers in a secure and timely manner it has fallen flat because of the incredible amount of upfront cost and effort and the need for all vendors to participate to provide any meaningful impact.

Hence the current solutions pursued by the Health Care technology industry has resulted in a difficult choice between care and privacy/economic fraud for patient. We see this issue greatly expanding as more and more mental health services are being delivered to individuals.

The new health care paradigm demands the need for effective and optimal care delivery for patients to yield better care outcomes. This requires that Principal Care providers are able to actively co-ordinate and collaborate with other care providers involved and ancillary health organizations like Labs and Pharmacy in care delivery.

In addition, given that many doctors don't want patients to access EHRs, it has resulted in patients adopting a passive role in tracking their health, and as a result feeling a lack of control and ownership of their health leading to patients becoming frustrated and being disengaged in their care. Though there has been a recent increase in Mobile Health Care apps helping individuals track their vitals and health parameters, the novelty has not translated to improved patient care or adherence and the expected positive outcomes as they too face the challenge of getting integrated into EHRs.

Another key impact of the new health care paradigm is the compensation model wherein the providers are eligible for receiving additional compensation beyond the basic compensation for the care delivered. This compensation is the result of savings that are generated based on how effectively the providers manage the care of the patient's health outcome. Any savings generated through efficient management of the patient's care can be retained by the providers and their network partners as part of the shared savings aspect of the new healthcare paradigm.

To realize these savings, a provider has to effectively track all the costs associated with the care of the patient and actively work with his partners to ensure timely health outcome. However, this requires that all the providers enter the care costs in near real-time while delivering care, which is very difficult to achieve based on the current EHR architecture. In addition, it is very hard for the principal care provider to divvy up the savings across the “key” provider partners to appropriately incentivize them to explore new care approaches. Though, the new healthcare policies provide the potential to incentivize providers to work together to improve care pathways, the current EHRs architectures fall short of enabling this ability.

One key aspect is the auditability of Care providers on two fronts. The first is the verification of whether the care provider actually delivered the care that he was obliged to deliver to the specification of the referring physician and at the same time the validation that the patient actually received it. Furthermore, in addition to the delivery of the care the financial expense incurred as part of this care should also be audited so as to ensure that care was appropriately paid and the charges were accurate. Tying the Care Auditability with the payment auditability provides the key advantage of reducing the significant fraud that currently plagues our healthcare system.

The key aspect to building a highly scalable and distributed Care Management system is a peer-to-peer architectural framework. Such a framework has already been used in a number of industry segments like, media, e-retail, supply-chain, etc. Furthermore, recent technologies involving the recordation of transfers of information in distributed ledger systems, like blockchain, have also enabled this framework to be adopted in other segments in which security is of prime concern like finance. Furthermore, it has been shown that such distributed ledger systems can easily be an add-on software connector to existing centralized frameworks. This has led us to explore using similar frameworks for their applicability in enabling a peer-to-peer framework for healthcare.

A system for recording the transfer of information in a distributed ledger system holds the promise of validating two or more entities engaged in a “healthcare transaction”. This provides two key attributes compared to a centralized authentication model. The first being that interested parties can engage with each other at a “transaction level” of “trust relationship”. The second is that the liability exposure in such a relationship is limited to only “transaction level” engagement. This is very useful as it limits the access of information and liabilities between parties involved and at the same time enables a party to get into a transaction relationship with a number of other providers based on their specific capabilities and type of care that needs to be delivered to the patient. This is significantly better than a conventional centralized systems needing to limit the number of providers for a wide range of patient needs due to the effort required to manage the access and associated liabilities. This is very much akin to Amazon being able to create a wide range of relationships with a variety of suppliers based on their customers' needs versus Walmart having to limit themselves to a limited number of suppliers.

Another key aspect of a peer-to-peer architecture is the ability to involve two or more parties in the validation of a transaction which may be necessary in the case of healthcare. A prime example is where a payor incentivizes a provider and a consumer in a three-way agreement to provide better compensation if they (provider, patient) jointly work together in reducing the overall cost of healthcare. Another alternative is when a primary provider engages another ancillary provider like a nutritionist to help train a patient to adopt a “low sodium” diet. Such a three-party agreement can also be validated by using blockchain technologies.

The new healthcare paradigm promises the opportunity for care providers as well as the patient to engage in a collaborative relationship that improves overall health of the patient and participate in the savings achieved. However, to effectively compensate all the key participants in a manner in-line with their contribution any contractual framework should be able to validate the milestones and their contributions. In a classical centralized data model this requires significant effort to manage such a contribution and as the number of parties involved increase the process becomes more complicated. In the case of a peer-to-peer framework using a blockchain as the validation model this is doable as smart contracts can be embedded in the blockchain. Furthermore, the fact that parties involved can also quickly monetize such an arrangement provides the added incentive for providers to engage in such arrangements.

Since blockchain uses personalized keys to validate transactions, any of the participants can ensure that only parties that are authorized have access to the patient health data. This avoids the “unintentional” leak of patient health data due to carte-blanch HIPAA releases that are currently signed by patients in order to receive care services. Thus the application of blockchain peer-to-peer frameworks enable the patients to have better control of their health data while providing access to those they deem necessary to be involved in their care. Furthermore, having such control also enables patients to provide “complete health information” about themselves in contrast to snapshots of information held in different systems. The keys benefits that result from the adoption of a blockchain based peer-to-peer framework are in the areas of fraud prevention, achieving high quality healthcare, affordable care, providing health care based on an individual's clinical and socio-environmental factors, and enabling adoption of a wellness lifestyle by the masses. We briefly elaborate on these benefits in the subsequent subsections.

Ensuring a framework that tracks and rewards patients and providers for taking ownership of care will drastically reduce the overuse and misuse of care services. By being able to track the care being delivered allows prevention of fraud and also hold both the patient and provider accountable for “validating” the care services being delivered at the stipulated quality in a real-time manner. This would significantly reduce the significant burden placed on payors, providers, and patients because of a few malicious healthcare participants.

The ability to seamlessly track and manage smart contracts in which the benefits can be redeemed with significant ease provides the necessary “carrot” for providers and patients to actively engage in a symbiotic collaboration. In contrast, if one or more participants tend to misbehave, appropriate penalties based on assumed liabilities can also be levied with similar ease. This “carrot/stick” approach we believe would provide the necessary push that is needed to shift the healthcare industry from a sickness management mindset to a wellness lifestyle mindset.

A key challenge is the inability of current frameworks to track “individual” impact on prescribed care plans. This framework enabling “individualized health information” to be easily accessed provide the necessary building blocks for the creation of real-time personalized care plans that are tailored based on an individual's clinical and socio-economic challenges. Furthermore, access to such an individual centric care plan also enables real-time correction of the plan resulting in a focus on prevention yielding to a path to sustained wellness.

Finally, through there has been a huge rise in self-care via the use of TeleCare devices like fit-bit, Apple/Samsung watch and vital monitoring devices. These devices have not been effectively integrated into the mainstream aspects of healthcare. The implementation of distributed ledger systems at such a low cost as that demonstrated by the newly emerging P2P payments using a modified version of blockchain implemented on chip cards and smart phones holds the promise that future TeleCare devices could come embedded with this capability, thus becoming an integral part of the peer-to-peer healthcare framework.

In this subsection we will discuss the long-term transformational impact that we believe could be possible with the implementation of peer-to-peer healthcare framework built on blockchain technologies.

Given that a blockchain based peer-to-peer healthcare framework has the potential of being able to formulate a complete horizontal patient health information (PHI) profile relatively inexpensively, it holds the promise of providing specific care plans and care regimes tailored to the individual's needs. This capability of generating complete PHIs holds the promise of eventually achieving personalized medical treatment for the masses.

The capability to track and validate specific care regimes that yield significant improvement in care outcomes combined with the fact that the care provider could be adequately compensated for his efforts would provide the necessary impetus to rejuvenate “care” innovations. Furthermore, the ability for a care provider to “license” his care methodology to any interested individual or care provider system via a peer-to-peer arrangement further holds the promise that entrepreneurs will pursue “innovative clinical” solutions that target a large number of individuals and will not be cost prohibitive for care regimes.

Another concern regarding the use of collected healthcare data is the accountability associated with who has accessed the data and for what purposes. Most healthcare data is gathered by a provider from the client, the patient. However, in many cases, the data is processed by a number of intermediaries including: laboratories, technicians, home healthcare workers and family members. Although there are privacy regulations associated with the disclosure of personal health information (e.g., the Health Insurance Portability and Accountability Act of 1996 (HIPPA)), many providers require that the client sign a release in order to provide this information to service providers. Service providers that are often given this healthcare data include: insurance companies, employers that are self-insured, pharmacy and drug companies, data processing companies and medical equipment manufacturers.

Hence the core focus of such a system is not a single entrant system, rather the focus is on a multi-entrant model. This underlying core difference is the basis of a new system in which the fundamental building block is a collaborative, distributed platform that requires at least two interacting parties in the validation of the result.

The core building block of a “person centered design” is a care collaboration that requires two or more parties involved in validation and authentication of the information that is being created. In addition, one of the parties involved must be the person who is the recipient of the care. Their involvement may be direct or indirect.

This resulting “double authenticated” data has the following benefits: i. a valid audit trail; and ii. creation of “actionable” data for other third parties that want to rely upon this data.

Any service provider that requests access to a person's data would require approval and validation from the individual.

The basis of a person-centered model is to ensure that any information that is entered into the system is authenticated and validated directly or indirectly by the person receiving care or their authorized representative.

Mode one: A service provider creates data based on an examination or test that was conducted or a set of information to which they have access. Mode two: An individual creates or logs data himself/herself or such data is created from a monitoring device. In case of Mode One, the information that has been entered needs to be authenticated by the individual through a care request from clinical or care personnel that he/she authorizes. In case there was a misdiagnosis or mistake, then an authorized representative of the person is able to amend the record working with the original creator of the data or, in extreme cases, can also reject or make that entry void.

In the case of Mode Two, any information that has been entered by an individual needs to be approved by authorized personnel before it can be used as valid information by other service providers.

A tagging process is embedded within the authentication process to create the necessary link of a particular data to a particular event. This ensures that information can be correlated. This tagging information can take a variety of forms that includes care plans, smart contracts with providers or individuals as well as other information that would provide context under which this data was gathered or generated.

Another aspect of ensuring adequate control of data is to know who has accessed that information. The system incorporates a twofold methodology that consists of: (i) a grant/revoke process; and (ii) an access notification process.

Through active participation, an individual would be more compliant with their care regime as well as more attuned to the outcomes. This allows patients to engage more productively with their healthcare service providers to augment healthcare services based on their individual characteristics. Furthermore, if the data is being collected, and is tagged for a personal milestone or some financial remuneration, the individual is more inclined to perform the actions required to trigger distribution of the incentive.

Given that the proposed system would create large volumes of valid data that could be correlated across multiple individuals, conditions and care regimes, the system would result in highly granular, deep longitudinal information that could allow for researchers and other interested parties to create much richer profile specific classes.

### BRIEF DESCRIPTION

The purpose of this summary is to present integral concepts in a simplified form as a prelude to the more detailed disclosure that is presented herein.

Disclosed herein are novel and useful systems, apparatus and methods for management of health and wellness information. Also disclosed is the use of such novel systems, apparatus, and methods for management of health and wellness information for management of health and wellness information transactions. It will be understood that management of health and wellness information transactions between multi-party entities may be performed by use of similar systems, apparatus, and methods for management of health and wellness information as disclosed.

Descriptions of certain illustrative aspects are described herein in connection with the annexed Figures. These aspects are indicative of various non-limiting ways in which the disclosed subject matter may be utilized, all of which are intended to be within the scope of the disclosed subject matter. Other advantages, emerging properties, and features will become apparent from the following detailed disclosure when considered in conjunction with the associated Figures that are also within the scope of the disclosure.

### BRIEF DESCRIPTION OF THE DRAWINGS

The disclosed subject matter itself, as well as a preferred mode of use, further objectives, and advantages thereof, will best be illustrated by reference to the following detailed description of embodiments of the device read in conjunction with the accompanying Figures, wherein:

FIG. 1 illustrates aspects of validating a proposed transaction involving multiple parties for management of health and wellness information according to some embodiments in a flowchart form.

FIG. 2 illustrates aspects of various steps involved in a transaction involving multiple parties for management of health and wellness information according to some embodiments in a flowchart form.

FIG. 3 illustrates aspects of the various participants, systems modules and interconnects involved in management of health and wellness information according to some embodiments in a block diagram form.

FIG. 4 illustrates the interconnects between the various system blocks in validation of a transaction involving multiple parties for management of health and wellness information according to some embodiments in a block diagram form.

FIG. 5 illustrates a method to record transaction data using reverse block chain according to some embodiments in a block diagram form.

FIG. 6 illustrates a method to create and update reverse block chain containing patient data according to some embodiments in a flowchart form.

### DETAILED DESCRIPTION

Reference now should be made to the Figures in which the same reference numbers are used throughout the multiple Figures to designate the same components. It will be understood that, although the terms first, second, third, etc. may be used herein to describe various elements, these elements should not be limited by these terms. These terms are only used to distinguish one element from another element. Thus, a first element discussed below could be termed a second element without departing from the teachings of the present disclosure.

The terminology herein is used to describe particular exemplary embodiments, and is not intended to generally limit disclosed embodiments and subject matter apart from reference to the Claims. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

One skilled in the art may apply the principles discussed herein to any computing environments and data communication networks. Further, one skilled in the art may apply the disclosed subject matter and principles discussed herein to data communication networks having any future physical configuration, layers, logical structures, and communications protocols, such as, for example, different or future generations or substitutes for the present-day Internet regime using wired and wireless channels and associated data communication protocols.

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, known methods, procedures, and components have not been described in detail so as not to obscure the implementations described herein. Also, the description is not to be considered as limiting the scope of the implementations described herein.

In the following detailed description, reference is made to the accompanying drawings that form a part hereof, and in which is shown by way of illustration specific implementations which may be practiced. These implementations are described in sufficient detail to enable those skilled in the art to practice the implementations, and it is to be understood that other implementations may be utilized and that logical, mechanical, electrical and other changes may be made without departing from the scope of the implementations. The following detailed description is, therefore, not to be taken in a limiting sense.

As referenced herein, “transaction” may mean an exchange of individual health and wellness information, to render health and wellness services, any other exchange of information between one or more parities for financial, research, correlation, audit, authentication, validation, verification, or any other such purpose, as well as a set of such individual transactions; “authentication” may mean the confirmation of the identity of a party to a transaction; “authorization” may mean the authority to, and in some situations the confirmation of the authority to, operate in a particular role as a party to a transaction; and “validation” may mean the confirmation of both authentication and authorization.

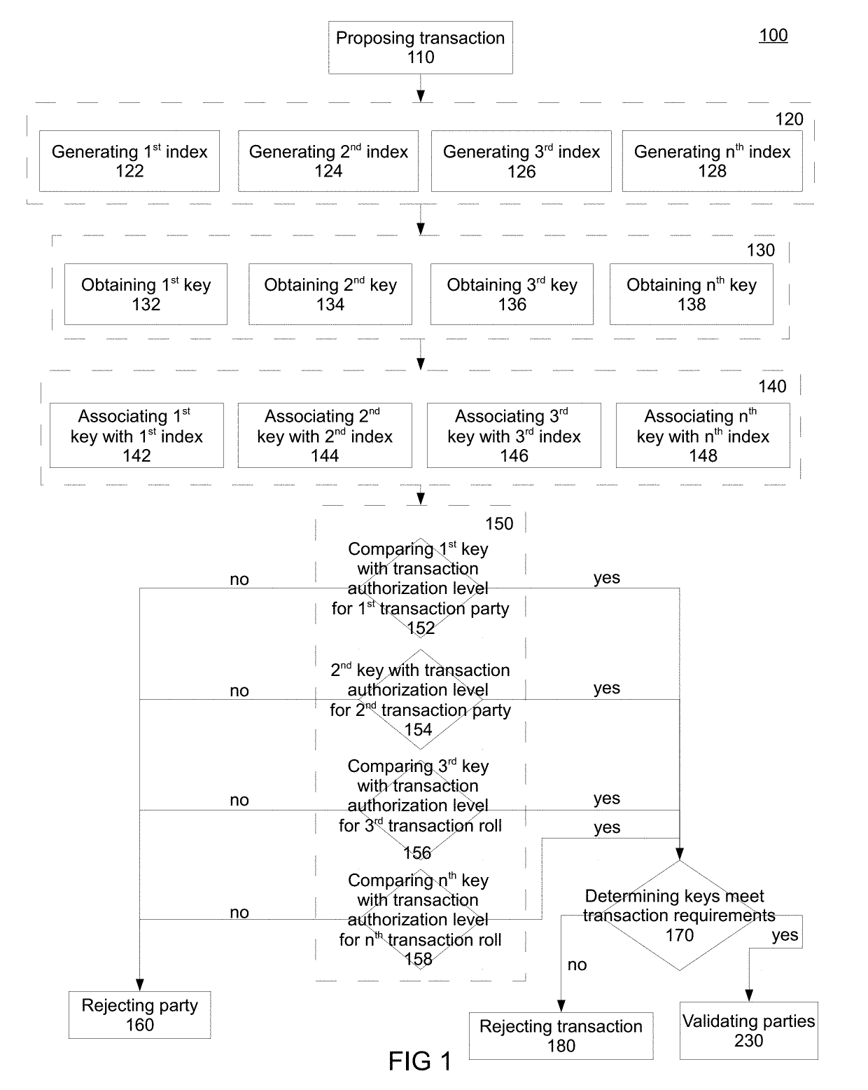

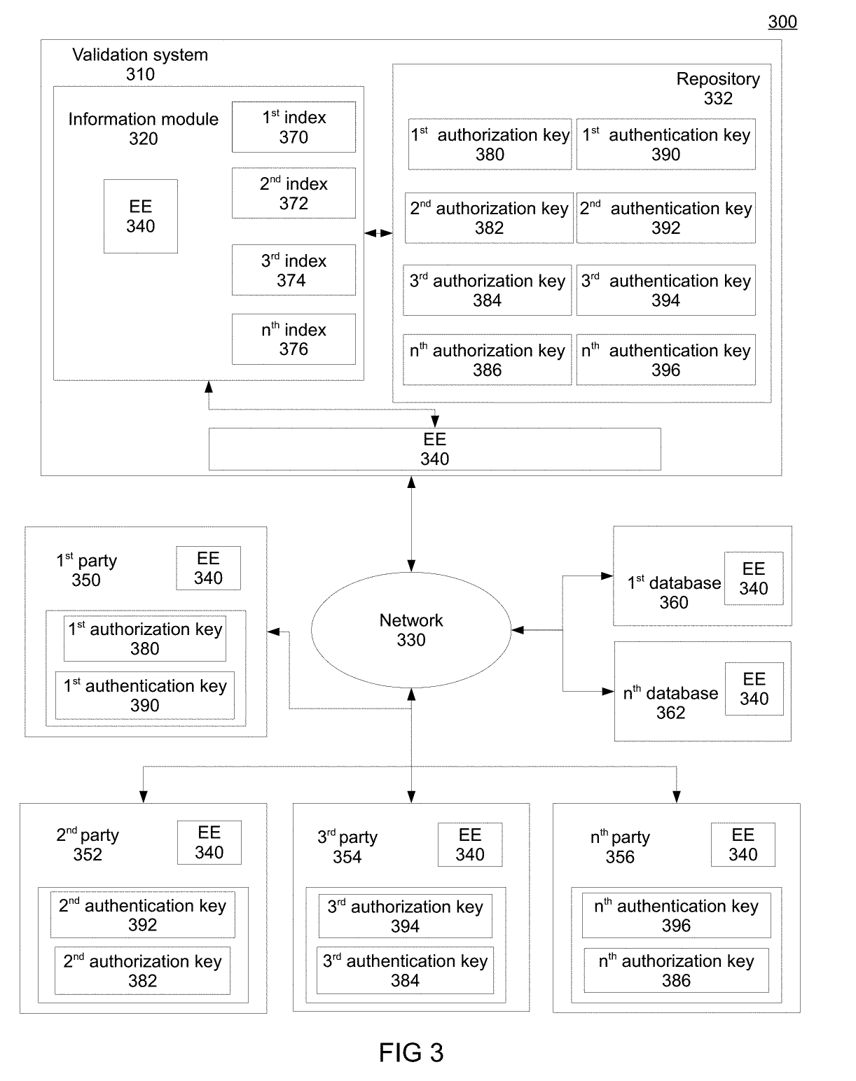

FIG. 1 illustrates a method 100 for validating a proposed transaction involving multiple parties for management of health and wellness information according to an exemplary embodiment. It will be understood by one of ordinary skill in the art that method 100 may be enabled or performed by operations of a suitable system for management of health and wellness information. It will be further understood that method 100 may be enabled or performed, for example, by operations of a system 300 for management of health and wellness information as illustrated in FIG. 3.

Referring again to FIG. 1, method 100 validating a proposed transaction involving multiple parties for management of health and wellness information may manage a transaction including health and wellness information. As referenced herein, “transaction with health and wellness information” may include, but is not limited to, transactions wherein, whether directly or indirectly, health or wellness information of an individual is accessed, referenced, retrieved, received, handled, read, considered, used, utilized, provided, held, registered, validated, checked, questioned, stored, secured, locked, introduced, produced, created, stored, changed, audited, corrected, approved, authorized, transacted, confirmed or communicated.

In an embodiment, such individual health and wellness information may be sensitive or subject to regulatory privacy requirements. Individual health and wellness information may include a spectrum of health and wellness information that may require compliance with one or more authorities, restrictions or standards. For example only, and without limitation, individual health and wellness information may include health and wellness information to be handled and managed in compliance with a statutory standard, such as the present HIPPA standard, or any different, or future, compliance standard for securing financial or health and wellness information.

Referring to FIG. 1, method 100 for managing health and wellness information may include proposing 110 a transaction having or requiring one or more of an authorization level, and an authentication level, in a health and wellness information management system. Proposing 110 may include receiving proposed transaction information for a proposed transaction such as, as an example and not as a limitation, the specific parties that need to be part of the transaction, the required authorization level for the transaction, the type of transaction, the required approval of the transaction, identification of a party that needs to approve the transaction, etc. A proposed transaction may include, or may require, identification and/or compliance with one or more of an authorization level, and an authentication level required for the transaction to be entered and completed, in a health and wellness information system. Proposing 110 may include references to indices identifying the proposed parties to the transaction.

Method 100 may include generating 120 at least three indexes, each corresponding to a different and distinct party, which may be party to a transaction. Each party may be qualified to perform or participate in such a transaction depending on one or more of their authorization level, authentication level, and one or more of an authorization level, and an authentication level required by the particular transaction. Each party may have certified eligibility to perform or participate in different transactions or in different roles for a transaction depending on their qualifications.

Generating 120 may include first generating 122 a first index. First generating 122 a first index may correspond to a first transaction party. Generating 120 may further include second generating 124 a second index which may correspond to a second transaction party, and a third generating 126 a third index which may correspond to a third transaction party. Each of the indexes may be associated with a different and distinct party to the transaction. The steps of generating 128 an index and associating the index with a party to the transaction may be iterated for (n) number indexes associated with (n) number of parties. As will be understood by one of ordinary skill in the art, an index is a link or pointer to a data structure that corresponds to the transaction at hand or to a transacting party. The data structure may include fields of information such as, as an example and not as a limitation, party identification, one or more of an authorization level, an authentication level, party role, category of service provided or consumed by the party, etc. Party identification may, as an example and not as a limitation, be a unique identifier for the transaction party. Authority and authentication level may, as an example and not as a limitation, be a value or other encoded identifier associated with a type of privileges the party has, such as read, audit, modify, validate, or create, with a security scheme utilizing mechanisms available in distributed ledger mechanisms, such as an example and not as a limitation, blockchain type environments or other analogous methods.

Method 100 may include obtaining 130 at least three keys, each corresponding to a different and distinct party, which may be party to a transaction. Each key may be associated with a party for the purpose of authentication of the party. As will be understood by one of ordinary skill in the art, a key maybe a unique identifier used to authenticate a transacting party. The key may include multiple fields of information such as, as an example and not as a limitation, identifier for authentication as well as an authorization level associated with a party. Authorization level for the party maybe a hierarchical value to represent the authority level of the party to designate whether a party may participate in a transaction by also assigning different authorization levels to various types of transactions according to some embodiment. According to another embodiment, the authorization level may be transaction specific and may authorize a party's role in the transaction itself such as, as an example and not as a limitation, the party's ability to propose a transaction, read or write access to certain subject data used in the transaction, party's authority to prescribe a treatment, party's ability to approve a proposed transaction, party's authority to audit, etc.

Obtaining 130 may include first obtaining 132 a first key. First obtaining 132 a first key may correspond to a first transaction party. Obtaining 130 may further include second obtaining 134 a second key which may correspond to a second transaction party, and a third obtaining 136 a third key which may correspond to a third transaction party. Each of the keys may be associated with a different and distinct party to the transaction. The steps of obtaining 138 a key and associating the key with a party to the transaction may be iterated for (n) number indexes associated with (n) number of parties. As will be understood by one of ordinary skill in the art, an authentication key could be implemented using a private-public key combination.

Method 100 may include associating 140 at least one of an authorization key, and an authentication key with each of the at least three indexes. In some embodiments associating 140 may include creating a pointer to a data structure that contains at least one of an authorization key, and an authentication key. Associating 140 may further include associating 142 a first at least one of an authorization key, and an authentication key with the first index. Associating 140 may further include associating 144 a second at least one of an authorization key, and an authentication key with the second index, and associating 146 a third at least one of an authorization key, and an authentication key with the third index. The step of associating 148 at least one of an authorization key, and an authentication key with an index associated with a party may be iterated for (n) number of authorization/authentication/audit keys associated with (n) number of indexes. In some other embodiments, the at least one of an authorization key, and an authentication key to be associated with an index may be passed along as a parameter in the request generating the transaction. In yet other embodiments, the at least one of an authorization key, and an authentication key may be obtained by utilizing multiple parameters or pointers to data structures in one or multiple data repositories, which may be controlled by the same or different parties. In yet other embodiments, the at least one of an authorization key, and an authentication key may be obtained by using the public-private key exchange with the parties involved in the transaction.

Method 100 may include comparing 150 at least one of the authorization key, and the authentication key of each index in the proposed transaction with at least one of an authorization level, and an authentication level required for participating in said transaction. Comparing 150 may include first comparing 152 at least one of the authorization key, and the authentication key of the first index in the proposed transaction with one or more of the authorization level, and the authentication level required for said transaction. Comparing 130 may include second comparing 154 at least one of the authorization key, and the authentication key of the second index in the proposed transaction with one or more of the authorization level, and the authentication level required for said transaction. Comparing 130 may include third comparing 156 at least one of the authorization key, and the authentication key of the third index in the proposed transaction with one or more of the authorization level, and the authentication level required for said transaction. Comparing 130 may include comparing 158 (n) number of authorization/authentication keys of (n) number of indexes with one or more of the authorization level, and the authentication level required for the proposed transaction.

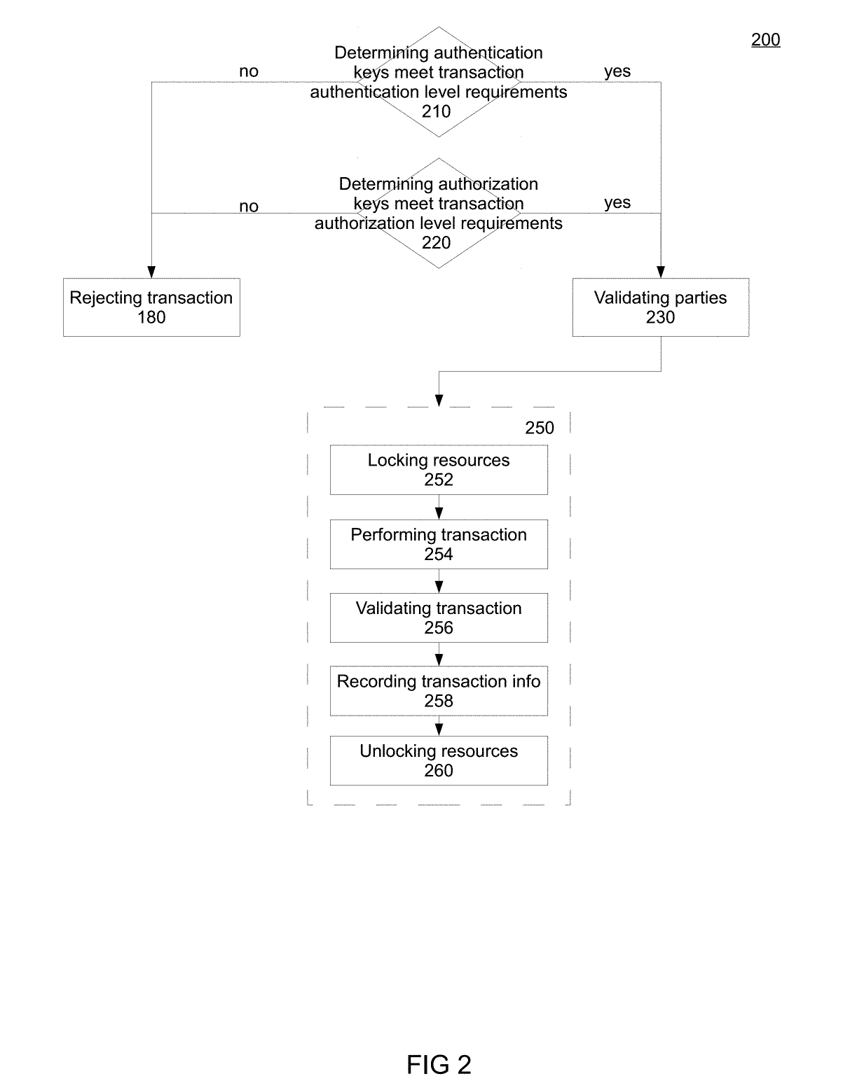

Method 100 may include determining 170 wherein at least one of the authorization key, and the authentication key associated with each of the potential parties to a proposed transaction are determined to be equal to, greater than, or less than the corresponding authorization/authentication level required to participate in the transaction. If one or more of the authorization/authentication keys submitted to the proposed transaction are determined to be less than the authorization/authentication level required by the transaction method 100 may comprise rejecting 160 the party associated with the deficient authorization/authentication key from participation in the transaction. If one or more parties are not rejected, the method 100 may comprise determining 170 whether the minimum required number of parties to the transaction have been determined to have sufficient authentication/authorization levels. If determining 170 results in there being insufficient parties to complete the proposed transaction, or the lack of a party filling a role necessary for the completion of the transaction, then the method 100 may comprise rejecting 180 transaction and the transaction may be terminated. If a sufficient number of authorization/authentication keys are determined to meet or exceed the authorization/authentication level required to participate in the transaction method 100 may proceed to validating 230 parties prior to the step of locking 250 an execution environment, which will be discussed in greater detail in reference to FIG. 2 herein below.

In embodiments each authorization/authentication key may be tailored to a variety of factors in order to ensure that the party associated with that particular authorization/authentication key is capable of entering into specific transactions, or fulfilling roles required for the completion of specific transactions, to which they are entitled. For example, a doctor may be provided with an authorization key that allows them to propose new treatments, propose modification of existent treatments, and reject proposed treatments, while a patient may have an authorization key that allows them to propose modification of existent treatments, and reject proposed treatments, but not to propose new treatments. Similarly, healthcare service providers may have their authorization keys limited to transactions or transaction roles in which they are qualified. For example, a dietician may have an authorization key that allows them to propose and reject transactions related to treatments involving dietary elements, but does not allow them to propose, reject, or even be party to transactions related to treatment involving physical rehabilitation, psychological treatments, or prescriptions (although with prescription transactions the dietitian's authorization key may allow for being a read only party so that they can ensure that any dietary treatment for which they are responsible does not conflict with a patient's prescriptions). By way of another example, health insurance providers may be provided with authorization keys that allow for the ability to indicate whether or not a transaction would be approved by the patient's insurance, but would not allow for acceptance or rejection of that transaction. Likewise, an insurance provider may possess an authorization key that allows them to participate in an audit transaction involving the patient that they are covering and service providers within their network, but does not allow them to audit transactions involving service providers who are not in their coverage network.

In embodiments different authorization/authentication keys may include further limitations to their ability to be a party to a transaction. Such limitations may include temporal limits, whereby said key may only be used for a limited time and will cease to be effective for providing access to some or all of the transactions for which it had previously been authorized after the expiration of a predetermined amount of time after generation or issuance.

FIG. 2 illustrates aspects of a method 200 for locking an execution environment (“EE”) according to embodiments. Method 200 may include determining 210 authentication keys meet the required authentication level for the transaction. Method 200 may further include determining 220 authorization keys meet the required authorization level for the transaction. Referring to FIG. 1, determining 210 authentication keys and determining 220 authorization levels maybe internal steps of determining 170 keys meet transaction requirements. If the determining 210 authentication keys meet the required authentication level and determining 220 authorization keys meet the required authorization level result in validating 230 parties, method 200 may include locking 250 an EE to restrict parties without proper, verified, authorization from access to the transaction, including access to health and wellness information associated or involved in the transaction. Locking 250 may enable only the specifically authorized parties to perform their action or function for the transaction during an authorized time period when the transaction is authorized to be performed between the specifically authorized parties. Locking 250 may prevent access to the accessible health and wellness information by all other parties until the lock expires or is terminated such as upon completing the transaction. It will be understood that locking 250 may encompass different authorized time periods and different accessible health and wellness information for each authorized party, and that such differences may reflect different authorizations for a party such as, for example, an administration authorization, or may reflect different required authorization levels for the transaction among parties otherwise having equal actual authorizations. For example, it will be understood that authorizations may be specified for a particular type of transaction, the timing of a transaction, particular accessible health and wellness information, and the role of a party.

Locking 250 or method 200 of generating a locked transaction may be effectuated through use of software or hardware assisted semaphore mechanisms. As an example, and not as a limitation, a software semaphore may include restricting access to a specific memory location or a software or hardware resource, which may have restricted access control by providing for the access of the memory space to generate a fault and the fault handler may take appropriate steps built around various rules such as temporal duration or scope of authorization, etc. to allow or deny access to the restricted memory space. Similarly, as an example and not as a limitation, a locking mechanism may be provided for by use of hardware that provides a protected execution environment which is preloaded with specific code and data that is only accessible to execute a program, or modify the stored data as a result of being provided the proper authorization and authentication keys of multiple parties involved in a transaction.

Locking 250 or method 200 of generating a locked transaction may include locking 252 the resources associated with the transaction. It will be understood that the locking 252 may be implemented by using distributed validation systems wherein the validation of the parties to a transaction and the transaction itself may be validated in different components of the system prior to locking the execution environment and/or the resources associated with the transaction. Once the resources for the transaction are locked locking 250 or method 200 of generating a locked transaction may include performing 254 the transaction. Locking 250 or method 200 of generating a locked transaction may further include validating 256 the transaction. Validating the transaction may involve verifying that the service provider has performed the services and the patient has received the services and has provided confirmation of the same. Locking 250 or method 200 of generating a locked transaction may include recording 258 the transaction and/or the parties involved in the transaction in a blockchain or analogous record. Once the transaction is performed and information about the transaction is recorded, locking 250 or method 200 of generating a locked transaction may include unlocking 260 the resources associated with the transaction. It will be understood that the unlocking 260 may occur upon completing the transaction, or upon expiration of the lock due to other factors such as expiration of a time limit associated with locking 252 or reduction of the authorization key of a party necessary to the transaction to a level less than the level of authorization required to participate in the transaction.

FIG. 3 illustrates aspects of system 300 for management of health and wellness information (or “health and wellness information system”) in accordance with some embodiments. System 300 may include a health and wellness information validation system 310 for secure management of health and wellness information. System 310 for management of health and wellness information may include information module 320 to aggregate the relevant information associated with all relevant parties that could be a party to a transaction. The information module 320 may further include data associated with first party in first index 370, data associated with second party in second index 372, and data associated with third party in third index 374. The index 376 associated with a party to the transaction may be iterated for (n) number indexes associated with (n) number of parties. Method 100 may be performed by operation or functioning of system 300. System 300 may include a health and wellness information repository module 332. The health and wellness information repository module 332 may include first authorization key 380 associated with first party, first authentication key 390 associated with first party, second authorization key 382 associated with second party, second authentication key 392 associated with second party, third authorization key 384 associated with third party, and third authentication key 394 associated with third party. The authorization key 386 and authentication key 396 associated with a party to the transaction may be iterated for (n) number of times with each such set associated with (n) number of parties respectively. The health and wellness information repository module 332 may include certain relevant information associated with each party that may be one of the parties to a transaction involving health and wellness data, along with rules, algorithms, blocks, and mechanisms to manipulate that data or operate on that data to identify specific actions or services that may authorized or restricted based upon multiple parameters associated with a party to the transaction or the transaction itself.

System 300 may include execution environment (or “EE”) 340. The EE 340 may be a secure area of a processing system. EE 340 may guarantee that code and data inside the processing system is protected with respect to confidentiality and integrity. Embodiments of EE 340 may include an isolated execution environment that may provide security features such as isolated execution, integrity of trusted applications along with confidentiality of their assets. In embodiments EE 340 may provide an execution space that provides a higher level of security than is provided in a typical processing system. As an example, and not as a limitation, EE 340 may be an isolated environment that runs in parallel with the standard processing systems, providing security for the secure environment necessary to perform transactions involving secure health and wellness data. EE 340 may provide secure mechanisms for transactions by using a hybrid approach that may utilize both hardware and software to protect data. Applications running or running in EE 340 may have access to the full power of a processing system's resources, while hardware isolation may protect these parties from actions of other parties that are not authorized to access the transaction, including information related to the transaction. Software and cryptographic isolation inside EE 340 may protect the trusted applications contained within the EE 340 from malicious activity that is outside of the scope of the authorization levels of the transaction or the authorizations enabled by the authorization keys of the parties to the transaction. EE 340 may be implemented by providing for such execution environment in a central location such as a server on a network, or it may be implemented by implementing a set of secure execution environments distributed across various parts of the system 300. It will also be understood that locking of the execution environment may be implemented by use of a distributed lock mechanism. It will be understood that EE 340 may be comprised of a processor embedded portion and an external portion distributed in other parts of the system 300. In other embodiments, the EE 340 may be implemented as an interconnect point between the modules of system 300 to ensure the trusted execution environment for all data and information exchange between the different sub-systems of system 300. As an example, and not as a limitation, FIG. 3 shows the various locations that the EE 340 may be implemented at. It is understood that one or more of such positioning of EE 340 in combination may be used to implement the system 300.

System 300 may include, a communications network environment or communications network 330 over which system 310 may communicate. Communications network 330 may enable packetized communications and logical connections between system 310 and (n) number of remote computing systems, devices, or parties 350, 352, 354, 356. Remote computing systems 350, 352, 354, 356 may include respective cooperating EE 340. Such remote computing systems 350, 352, 354, 356 may include, for example, but are not limited to, servers, nodes, peer devices, client portals, laptops, desktops, handheld computing devices such as smartphones or tablet computers, and communications enabled smart devices, such as wearable technology enabled devices or medical diagnostic or treatment devices, commonly referenced as IoT-devices. In embodiments, remote computing systems 350, 352, 354, 356 may comprise a source machine from which data is generated/transmitted, a destination machine or system which receives transmitted data, or another suitable relationship may be structured and included.

Communications network 330 may enable packetized communications and logical connections between system 310 and one or more (n) remote medical information databases 360, 362. The remote medical information databases 360, 362 may include a respective cooperating EE 340. Said remote medical information databases 360, 362 may provide information related to transactions that are to occur within EE 340 to system 310 and/or remote computing systems 350, 352, 354, and 356 through their respective cooperating EE 340. Similarly, transactions within EE 340 and information related thereto may be transmitted via communications network 330 to said remote medical information databases 360, 362, through their respective cooperating EE 340. It is understood that in embodiments with EE 340 being implemented in a centralized location, the system maybe implemented by using encrypted data and packetized communication between the remote devices/systems and the medical and health care system with all secure execution and use of unencrypted data happens in the centralized EE 340.

System 300 may include software applications, programs, or modules instructing an operating system to perform tasks such as instructions, steps, facilitating requests, system maintenance, security, data storage, data backup, and execution of algorithms. The provided functionality may be embodied directly in hardware, in a software module executed by a processor, or in a combination of the two. Furthermore, software operations may be executed, in part or wholly, by one or more servers or a client system, via hardware, software module, or a combination of the two. A software module, application, or executable may reside in accessible RAM, ROM, EPROM, EEPROM, flash memory, registers, hard disk, removable disk, CD-ROM, DVD, optical disk, or any other form of storage medium, and may be implemented using a local storage, network storage, or utilizing cloud storage services. An exemplary storage medium may be coupled to a processor such that the processor can access, read information from, and write information to, the storage medium. In the alternative, the storage medium may be integral to the processor. Portions of the processor and storage medium may also reside in an application specific integrated circuit (ASIC).

In embodiments EE 340 may be centrally located execution environment on a single system, such as system 310. In such embodiments, information may be inputted into centralized EE 340 from a plurality of remote computing systems, such as remote computing systems 350, 352, 254, 356 and/or medical databases 360, 362, and even system 310, but all resources locked down for the one or more of authorization, authentication, auditing, validation, and/or processing and recording of the transaction may be solely within centralized EE 340.

In embodiments EE 340 may be a distributed execution environment with portions of EE 340 located on different devices that may be party to a transaction. For example, referring to FIG. 3, a transaction involving remote computing systems 350, 352, 354, and 356 may utilize an execution environment that shares resources from more than one of EE 340 and one or more of cooperating EE 340 which are implemented in the remote devices themselves.

In embodiments a system or systems using an EE allows parties involved in transactions of sensitive medical information to reliably and securely control, audit, validate, bill for, and otherwise use that information. In embodiments this may be achieved by the EE being an atomic system, an atomic operating environment, or may otherwise utilize atomic processing, as will be understood by one having skill in the relevant art.

In embodiments the EE 340 may use special purpose tamper resistant secure processing units to provide security for the transactions occurring therein, including the storage of transaction information and the communication of transaction information between systems involved in the transaction.

In embodiments system 310 may include a memory and a processor. The memory may store indexes, the information including authorization/authentication keys associated therewith, and other party account information.

In embodiments the processor may be operable to receive a request from a party to perform a transaction. The processor may also be operable to determine a number of validations required to confirm the transaction based on the authorization/authentication level(s) associated with the transaction. In embodiments transactions may require not only a specific number of validations require to be confirmed, but each of those validations required may have a different validation level.

In embodiments system 310 may provide various notifications to various remote computing systems, such as confirmation of receipt of an authenticated transaction proposal, a request for participation in a transaction, confirmation of completion of a transaction, and notice of a failure of a transaction. Such notifications may be transmitted via communications network 330.

In embodiments system 310 may communicate validation data as part of a transaction including an audit transaction. Validation data may include validated tokens, credentials, authorization/authentication keys, and any other suitable data that may be used to confirm that the transaction and the parties participating in the transaction are authorized or otherwise valid.

In embodiments wherein one or more of the remote computing systems involved in a transaction through the EE is an IoT medical monitoring and/or treatment device, the transaction, when completed, may include providing instructions to the IoT device that may cause the IoT device to perform actions in the real world. Such real world actions may include executing a monitoring action or providing medical treatment. In some embodiments the transaction may cause the IoT to deviate from a prior programmed treatment.

An example IoT medical treatment device may be an insulin pump that a patient uses to treat their diabetes. The insulin pump may be originally programmed to deliver insulin at a basal rate of 0.4 units/hour, but the patient still experiences consistently high blood sugar levels so they talk to their doctor about modifying the insulin dosage. If the doctor agrees that the patient could benefit from a modification of their basal rate insulin dosage then they may submit a proposal for a transaction involving a modification of the patient's basal insulin dosage (from say 0.4 units/hour to 0.6 units/hour) along with their authorization key to the system, Assuming the system validates that the doctor's authorization key is sufficient to propose said transaction, the patient may review the transaction request through the system, and if suitable may confirm the proposed treatment modification transaction using their own authorization key. The insulin pump may also be sent a notification of the dosage modification transaction and respond to the system with its own authorization key. If the insulin pump's authorization key indicates that it is capable of, and has the authorization to, increasing the dosage to 0.6 units/hour in accordance with the transaction request, the system approves and records the transaction, and transmits instructions to the insulin pump to modify its treatment profile from its prior programmed treatment of 0.4 units/hour to the authorized and validated treatment of 0.6 units/hour. The insulin pump may then perform the updated treatment profile responsive to receipt of said authorized and validated instructions. Accordingly, this system may be used to effectuate tangible actions related to medical care treatment in the real world based on authorized, corroborated, and validated transactions of information.

In embodiments a validation system, such as system 300 or system 310 may validate parties to a transaction and/or the transaction itself, including completion of the transaction through suitable validation means, such as by comparing one or more authorization and/or authentication keys submitted to the system with one or more levels required by the transaction. In embodiments, a transaction may be rejected by any one or more parties to the transaction depending upon the authorization keys of the parties.

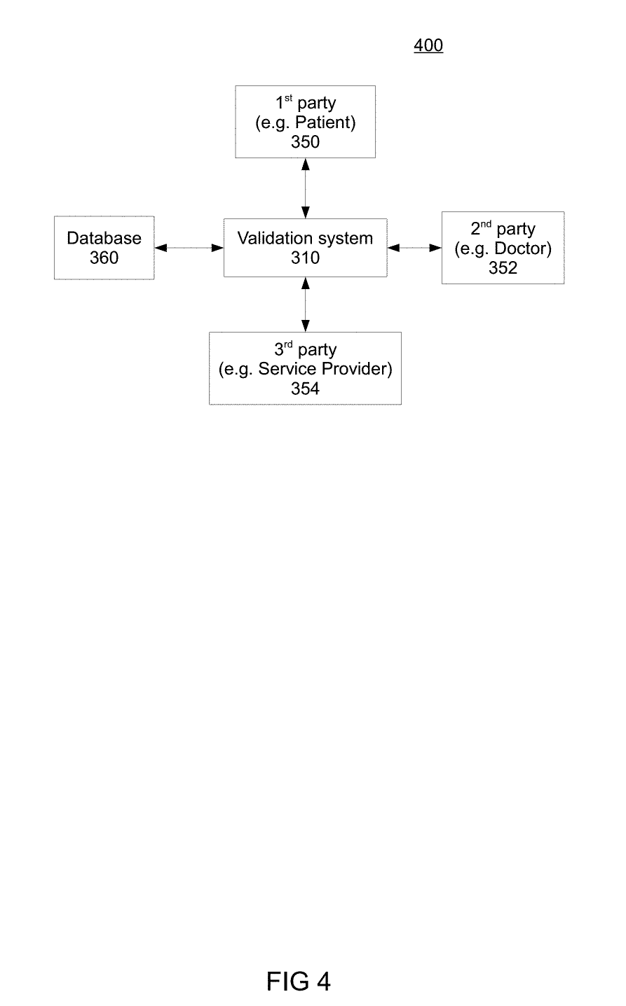

FIG. 4 illustrates a system 400 that requires two or more parties 350, 352, 354 involved in validation and authentication of information being created, according to embodiments. In the embodiment depicted in FIG. 4 the parties involved are a patient 350, the patient's doctor 352, and a service provider 354. In this embodiment doctor 352 may submit a request consisting of a treatment for the patient 350, wherein said treatment may be provided by the service provider 354. In order to be a valid treatment the proposed treatment must be within the authority level associated with the doctor 352. Once the proposed treatment submitted by doctor 352 is transmitted to validation system 310, validation system 310 confirms that said prescription of said treatment is within the doctor's authorization level by comparing the authorization required for the treatment (i.e. the transaction) with the authorization key that the doctor 352 submits with the transaction request. If the doctor's authorization key meets or exceeds the authorization level required to submit the treatment proposal transaction, validation system 310 may request confirmation of the proposed treatment from the patient 350. Before the treatment is entered into the patient's healthcare record it must first be confirmed by the patient 350. This patient confirmation must also be within the authority level of the patient 350 (although, generally the patient's authority level will extend to confirmation or rejection of just about every element associated with their healthcare record). Once the patient 350 responds to the treatment confirmation request, the validation system 310 may validate the patient's response by comparing the patient's authorization key with the authorization level required to confirm the proposed treatment. If the patient's authorization key meets or exceeds the authorization level required to confirm the treatment, validation system 310 validates the patient's confirmation. Once the proposed treatment's submission by the doctor 352 is validated by validation system 310 and patient's confirmation of the treatment is validated by validation system 310, validation system 310 enters the treatment transaction into the patient's healthcare record and records the transaction and its associated information. In embodiments where the validated treatment is capable of being fulfilled by a service provider service the service provider 354 may be provided with an index containing authorization keys commensurate with the authorization levels for transactions in which service provider 354 is qualified to engage. In embodiments any one of the parties 350, 352, 354 may propose a transaction for the service provider 354 to provide a service to the patient 350. No matter which party suggests said transaction the service provider 354 and the patient must submit requests to enter into said transaction through validation system 310 (it does not matter whether the service provider 354 or the patient submits the request first). Validation system 310 validates both that service provider's request and patient's request authorization keys equal to or greater than the authorization level required to enter in the transaction. If both authorization keys are validated by validation system 310 then validation system executes the transaction and records the transaction and its associated information. If one or both authorization keys are not validated by the validation system 310 due to them not meeting the requirements of the authorization level associated with the transaction, validation system 310 rejects the transaction. In embodiments such a service transaction may be an initial engagement between service provider 354 and patient 350, or a transaction directed at confirming an action taken during the course of service provider's providing of service to patient 350 (e.g. that patient 350 performed their weekly rehabilitation task as assigned by the treatment that was previously suggested by doctor 352, confirmed by patient 350, and validated by validation system 310). If, for example, the transaction between the service provider 354 and patient 350 was related to the completion of a rehabilitation task required by doctor's validated treatment and was properly validated by validation system 310, doctor 352 may access patient's healthcare record and see that validation system 310 validated the completion of said task, and may rely on that information more than they would be able to rely on non-validated information, or information provided by only service provider 354 or patient 350.

In embodiments validation system 310 may be used to validate and record transactions between more than three parties as long as at least the minimum number of parties having the minimum required authorization keys for the transaction are involved in the transaction through validation system 310.

In embodiments one or more parties to a proposed transaction may be notified by validation system 310 once the proposed transaction has been submitted to and verified by validation system 310.

In embodiments validation system 310 may be accessed by a party with a suitable authorization key to audit transactions that had been previously run through validation system 310. In order to do this the auditing party submits a request to audit a transaction to validation system 310 along with their authentication/authorization key and validation system 310 compares the authentication/authorization key with the authentication/authorization level of the transaction that the party is requesting to audit. If the authentication/authorization key meets or exceeds the authentication/authorization level of the transaction then validation system 310 retrieves the information related to said transaction that had been recorded at the time that the transaction was performed and validated and transmits said information to the requesting party. In embodiments validation system 310 may record information related to the audit transaction, including the requesting party, the information provided to the requesting party, and other information related to the audit (e.g. time of audit, IP address of auditing party, etc.).

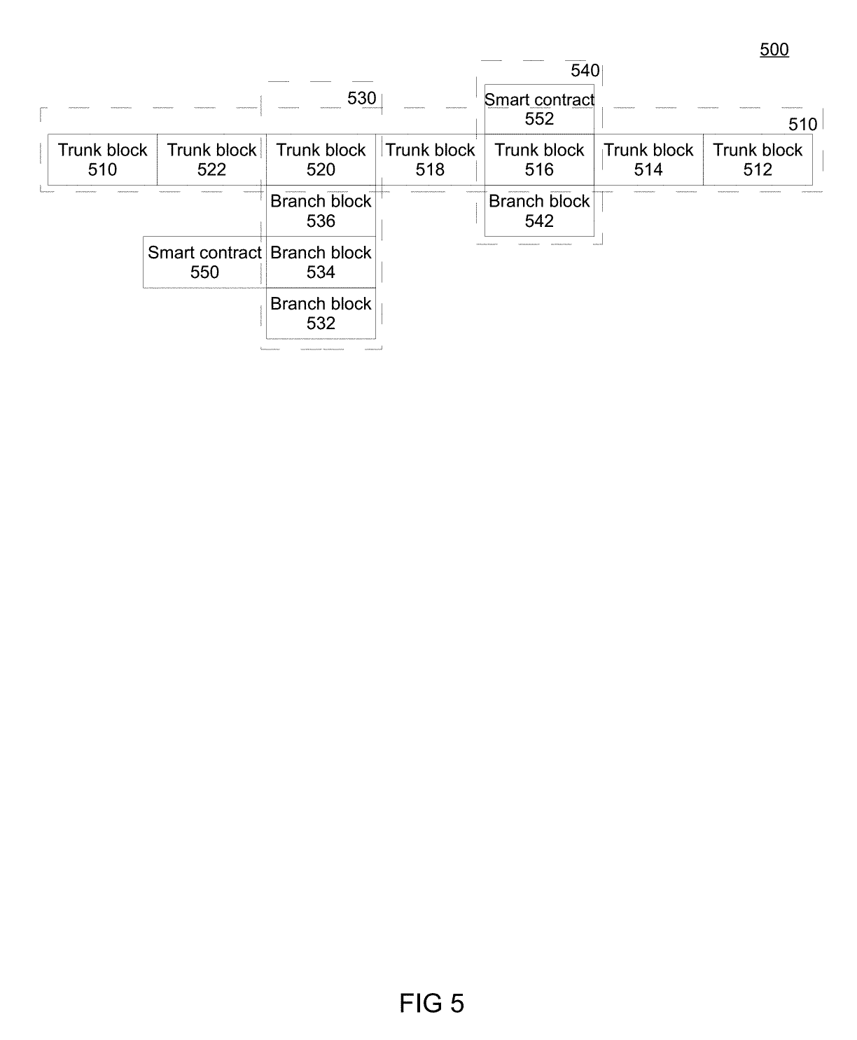

FIG. 5 illustrates an exemplary reverse tree record 500, which may be recorded in a distributed ledger system, for maintaining and updating a patient's medical and wellness information in a verified manner. Reverse tree record 500 may comprise a trunk 510 and one or more branches 520, 530. Trunk 510 may comprise one or more trunk blocks 512, 514, 516, 518, 520, 524. Each branch 530, 540 may comprise one or more branch blocks 532, 534, 536, 542. Each trunk block (e.g. trunk block 520) may have one or more branch blocks 532, 534, 536 associated therewith. Smart contract 550, 552 may be associated with one or more branch blocks.

In embodiments each trunk block 512, 514, 516, 518, 520, 524 may be associated with a different modality, which may be related to an aspect of a patient's health, care, and/or wellness. A modality may include, for example, an injury, an event (such as a car wreck), a treatment, a diagnosis, or a body part. A branch block 530, 540 may be created responsive to a validation system (such as system 310) validating a transaction. If the validated transaction is related to a modality represented by an already existing trunk block then the branch block representing said validated transaction may be associated with the related trunk block at a point closest to the trunk block. If the validated transaction is related to a modality that is not yet represented by an existing trunk block, then the branch block representing said validated transaction may cause a new trunk block for the modality associated with the validated transaction may be created and appended to the existent trunk.

In embodiments reverse tree record 510 may be generated by systems for secured and verified transactions, such as system 300. Additionally, embodiments of reverse tree record 510 may be generated by, or responsive to, methods for management of health and wellness information, such as method 100.

In embodiments the creation of a new branch block associated with a trunk block already having at least one already existent branch block associated therewith may cause the prior-existing branch block to be archived. This archiving of branch blocks occur automatically if the newly created branch block (or a plurality of branch blocks including the newly created branch block) meets all requirements for a rule set associated with the prior-existing branch block to become archived. For example, if a patient has a right shoulder injury, and the patient and doctor submit a treatment for said shoulder injury consisting of five rounds of physical therapy to the verification system, the system may verify the treatment transaction and record that verification as a branch block in the patient's reverse tree record. At this point the patient's reverse tree record may comprise a trunk block whose modality consists of something along the lines of “right shoulder”, with a single branch block saying “treatment—five rounds of rehabilitation”. After the patient completes each session of rehabilitation with verified rehabilitation service provider the patient and service provider submits a transaction consisting of completion of a single round of rehabilitation to the verification system and, upon verification of each one of such transactions the system creates a new branch block under the “right shoulder” trunk block, between the “right shoulder” trunk block and the previous branch block thereunder. After there have been five verified transactions reflecting five completed rounds of rehabilitation for the shoulder, on the addition of the fifth rehabilitation branch block to the “right shoulder” trunk block, the initial “treatment—five rounds of rehabilitation” branch block may be automatically archived. Alternatively, the creation of a new branch block may prompt a verified party (e.g. the doctor) to tell the system whether or not a prior-existing branch block should be archived.

In embodiments a transaction may comprise a request to close a modality. This may, for example be the doctor and patient submitting and corroborating that an injury is cured, a course of treatment has been completed, a diagnosis has been found to be inaccurate, etc. Upon verification of the transaction to close the modality the system may create a branch block for said modality indicating validation of the transaction associated therewith, and may archive the branch with which it is associated along with its associated trunk block.

In embodiments archived branch blocks and trunk blocks may not be used as relevant factors for a patient's current health status, but may still be available and used as for analysis of the patient's medical history, or for use in health studies, or as an audit trail.

In embodiments a transaction as discussed hereinabove, may comprise or consist of a patient's reverse tree record, or portions thereof.

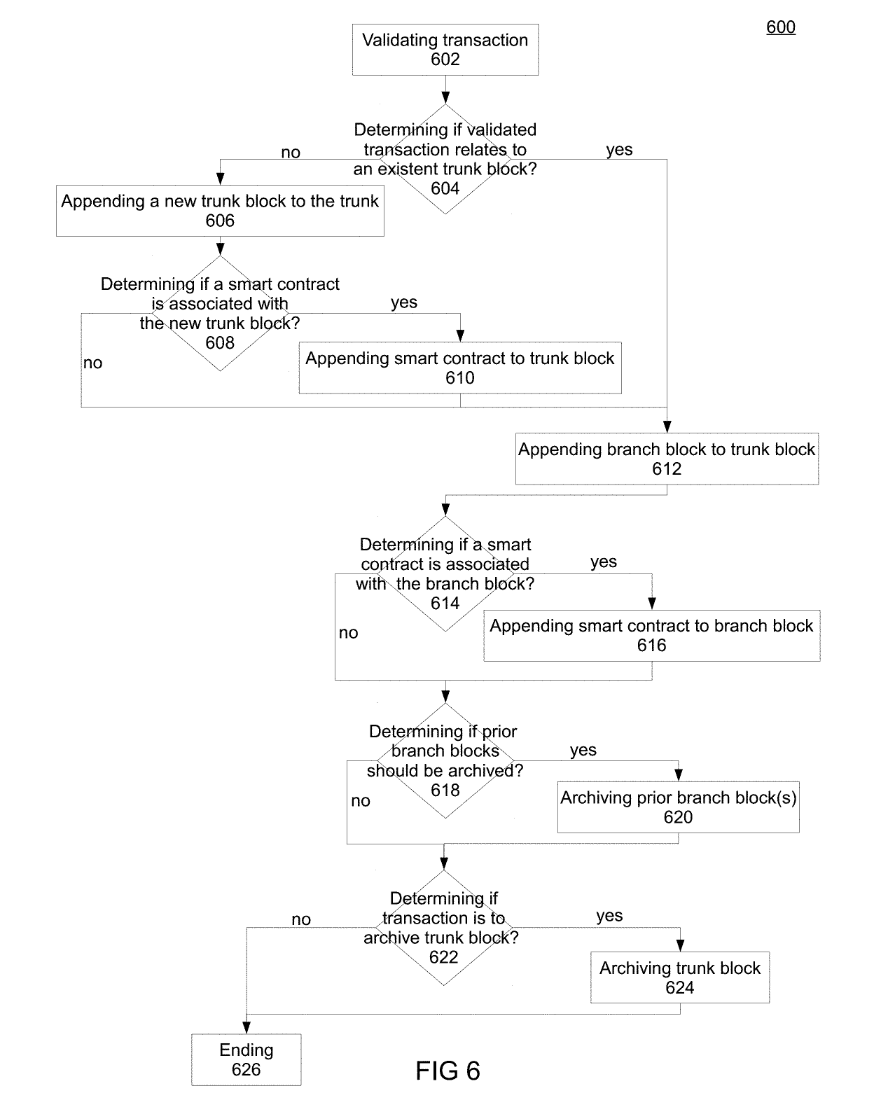

In embodiments a patient's reverse tree record, or portions of a patient's reverse tree record, may be associated with one or more smart contracts (like smart contract 550, 552). Such smart contracts may outline specific goals that need to, or may be achieved by parties to the patient's healthcare treatments. Such goals, for example, may include the prevention of negative health outcomes, or completion of treatments or treatment programs that would result in an incentive being earned by one or more of the patient, the patient's healthcare provider, and the patient's healthcare service provider. In embodiments, parties involved in validation of the transactions that create and populate a patient's reverse tree record, as described hereinabove, may also be required to validate all associated payments, or other elements, outlined in the smart contract. Upon validation of all transactions required to trigger the vesting of an option in the smart contact, the vesting may occur and, for example, the patient or provider may be compensated accordingly. FIG. 6 illustrates a method 600 of creating and updating a reverse tree record, such as reverse tree record 500, according to embodiments. Method 600 includes validating 602 a transaction. Validating 602 may be performed by a validation system such as distributed validation system 300 or consolidated validation system 310. Once the transaction is validated pursuant to validating 602 method 600 may include determining 604 if the validated transaction relates to an already existent trunk block or modality represented by such a trunk block. If determining 604 finds that the transaction is not related to an existent trunk block method 600 may include appending 606 a new trunk block having a modality associated with the validated transaction to the trunk. If a new trunk block is appended to the trunk method 600 may include determining 608 if a smart contract is associated with the new trunk block. If a smart contract is associated with the new trunk block method 600 may include appending 610 said smart contract to the new trunk block. If there is no smart contract associated with the new trunk block, or if determining 604 indicates that the validated transaction is related to an already existent trunk block method 600 may include appending 612 a branch block related to the transaction to either the new trunk block, or to the existent trunk block with which the transaction is associated. In embodiments appending 612 may consist of attaching the newly created branch block to its associated trunk block, and if prior existing branch blocks were already attached to that trunk block, then placing the new branch block in-between the trunk block and the prior-existing branch blocks. Once the branch block is appended to a trunk block method 600 may include determining 614 if a smart contract is associated with the newly appended branch block. If determining 614 identifies a smart contract that is associated with the new branch block method 600 may include appending 616 said smart contract to said new branch block. Once the smart contract is appended to the new branch block, or if determining 614 does not identify a smart contract associated with the new branch block, method 600 may include determining 618 if any prior-existing branch blocks associated with the trunk block with which the branch block associated with the validated transaction should be archived. If determining 618 indicates that one or more prior-existing branch blocks should be archived method 600 may include archiving 620 said one or more prior-existing branch blocks. In embodiments determining 618 may be rule based, and in alternate embodiments determining 618 may be responsive to an input from an authorized party. After archiving 620 or if determining 618 indicates that any prior branch blocks should not be archived, including if no prior branch blocks exists, method 600 may include determining 622 if the verified transaction is to archive the trunk block with which said transaction is associated. If determining 622 indicates that the verified transaction is to archive the associated trunk block, such as, for example the transaction is an indication that the modality represented by the trunk block has been cured, or is otherwise no longer pertinent to the treatment of the patient with which the reverse tree record is associated, method 600 may include archiving 624 of said trunk block. Once archiving 624 of the trunk block has been completed, or if determining 622 indicates that the transaction is not designed to archive the trunk block then method 600 may terminate via ending 626.

Methods disclosed herein may be performed in any suitable order unless otherwise indicated herein or otherwise clearly determined by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein.

The detailed description set forth herein in connection with the appended drawings is intended as a description of exemplary embodiments in which the presently disclosed apparatus and system can be practiced. The term “exemplary” used throughout this description means “serving as an example, instance, or illustration,” and should not necessarily be construed as preferred or advantageous over other embodiments.

Further, although exemplary devices and figures to implement the elements of the disclosed subject matter have been provided, one skilled in the art, using this disclosure, could develop additional hardware and/or software to practice the disclosed subject matter and each is intended to be included herein.

In addition to the above described embodiments, those skilled in the art will appreciate that this disclosure has application in a variety of arts and situations and this disclosure is intended to include the same.

What is claimed is:

1. (canceled)

2. (canceled)

3. (canceled)

4. A system for secure and verified exchange of information comprising:
a memory operable to store a plurality of transaction requests comprising:
an authorization key;
an authentication key; and
a proposed transaction;
a processor communicatively coupled to the memory;
the memory including executable instructions that upon execution by the processor cause the system to:
store transaction requests received by the system in the memory;
select a proposed transaction received from a received transaction request as a create transaction request, and identify the transaction request selected as a create transaction request as a transaction party;
identify a received transaction request comprising an authorization key that meets a required authorization level and an authentication key that meets a required authentication level of the create transaction request as transaction party;
lock resources responsive to identification of the transaction parties;
perform the proposed transaction within the resources;
record performance of the proposed transaction in the memory; and
unlock the resources.

5. The system of claim 1; wherein the proposed transaction comprises at least one of the list consisting of:
creating a transaction
joining a transaction
viewing a transaction; and
auditing a transaction.

6. The system of claim 2, wherein a proposed transaction comprising creating a transaction additionally comprises:
transaction terms;
a transaction identifier;
a required number of transaction parties;
an authorization level; and
an authentication level.

7. The system of claim 1, wherein the system for secure and verified exchange of information is distributed between a plurality of devices.

8. The system of claim 1, wherein the resources comprises a secure data execution environment.

9. The system of claim 5, wherein the data execution environment is a semaphore.

10. The system of claim 6, wherein the secure data execution environment is an atomic execution environment.

11. The system of claim 1, wherein the resources are distributed between a plurality of devices.

12. A method for secure and verified exchange of information between devices comprising:
receiving a proposed transaction;
generating a plurality of indexes;
obtaining a plurality of keys; each of the keys associated with an index;
determining whether an index meets requirements for the transaction, said determining comprising:
determining if a key associated with the index meets an authorization level for the transaction; and
determining if a key associated with the index meets an authentication level for the transaction;
validating indexes that receive a positive determination that the index meets the requirements for the transaction as transaction parties;
locking resources responsive to the validation of a number of transaction parties equal to a required number of transaction parties performing the proposed transaction within the locked resources;
validating the performance of the proposed transaction;
recording information related to the performance of the proposed transaction; and
unlocking the resources.

13. The method of claim 9, wherein the required number of transaction parties is specified by the proposed transaction.

14. The method of claim 9, wherein the required number of transaction parties is at least three.

15. The method of claim 9, wherein at least three indexes are generated.

16. The method of claim 12, wherein an index is associated with one of the group consisting of:
a patient;
a doctor; and
a service provider.

17. A blockchain system for managing and archiving data comprising:
a trunk comprising:
a plurality trunk blocks, each of the plurality of trunk bocks associated with a modality;
a branch associated with a trunk block, the branch comprising:
a branch block, wherein the branch block comprises information related to the modality of the trunk block with which the branch block is associated, and wherein the branch block is submitted with a first private key and verified via a second private key associated with the first private key.

18. The blockchain system of claim 14, further comprising a smart contract associated with at least one of a trunk block, a branch, and a branch block.

19. A method of managing and archiving data in a blockchain comprising:
validating a transaction;
determining if a modality to which the validated transaction is associated is represented by a trunk block;
if the modality is not represented by a truck block, creating a trunk block representing the modality; and
appending the validated transaction to said trunk block representing the modality as a new branch block.

20. The method of claim 16, further comprising:
determining if prior branch block(s) should be archived responsive to the new branch block; and
archiving said prior branch block(s) responsive to a positive determination.

21. The method of claim 16, further comprising:
determining if the trunk block should be archived responsive to the new branch block; and
archiving the trunk block and all branch blocks related thereto responsive to a positive determination.

22. The method of claim 16, further comprising:
determining if prior branch block(s) should be invalidated responsive to the new branch block; and
invalidating said prior branch block(s) responsive to a positive determination.
